(19) United States
(12) Patent Application Publication
(10) Pub. No.: US 2008/0039734 A1
(43) Pub. Date: Feb. 14, 2008

(54) Method for measuring airway resistance

(75) Inventors: Joseph Lomask, Wilmington, NC (US); Morton Lomask, Sharon, CT (US)

Correspondence Address: WILLIAM J. MASON;MACCORD MASON PLLC, POST OFFICE BOX 1489, WRIGHTSVILLE BEACH, NC, 28480, US

(73) Assignee: Buxco Electronics, Inc.

(21) Appl. No.: 11/502,944

(22) Filed: Aug. 11, 2006

(51) Int. Cl. A61B 5/08

(52) U.S. Cl. 600/529

(57) ABSTRACT

A method of characterizing the respiratory properties of a conscious living organism from a single respiratory waveform containing thoracic and nasal flow signal components is described that includes acquiring a single box flow waveform containing thoracic and nasal flow signal components, measuring the areas of peaks of the waveform, and characterizing respiratory properties from the peak areas. A method is also described for characterizing the respiratory function of a conscious living subject by acquiring separate thoracic and nasal respiratory waveforms, determining the phase shifts between the waveforms at first and second time spaced points, determining the net inspired volume between the points, and characterizing respiratory function using the phase shift and net inspired volume.

### BACKGROUND OF THE INVENTION

(1) Field of the Invention

The present invention relates to the evaluation of respiratory function of a conscious living organism from characteristics of respiratory flow signals, and in particular to the calculation of airway resistance from a single combined waveform instead of from separate thoracic and nasal waveforms. The invention also relates to improved calculation of airway resistance from separate thoracic and nasal waveforms.

(2) Description of the Prior Art

In the traditional and direct measurement of respiratory resistance, i.e., humans, small and large animals, two signals are measured separately, and compared. One configuration measures lung pressure versus the air flow in and out of the subject's mouth and nose. Another configuration measures the airflow in and out of the subject's mouth and nose versus the rate of change of the chest (thoracic) volume. For example, U.S. Pat. No. 6,287,264, issued Sep. 11, 2001, and U.S. Pat. No. 6,723,055, issued Apr. 20, 2004, both to Hoffman, describe methodology for the measurement of bronchoconstriction and other obstructive disorders in which the thoracic movement, as an indicator of lung volume, and nasal flow of a test animal subject are measured.

Nasal flow measures the flow of air at the nose or mouth. The air entering the nose is at room temperature and humidity conditions, and when exiting it is at close to animal body conditions. On the basis of temperature and humidity alone, the inspired volume will be smaller than the expired volume, as long as the room temperature is cooler and dryer than body temperature and humidity. Also, any perceived change in volume due to metabolism also affects the expired volume when compared to inspired volume. Generally speaking, volume changes due to metabolism are very small compared to the typical inspired volume of the subject. However, no change due to internal lung pressure is detectable.

The thoracic flow measures the respiratory flow by measuring the chest expansion and contraction. This measurement is somewhat indirect, and as such, does not actually measure the flow of air into and out of the animal. For example, if the airway is occluded so that no air can flow into or out of the nose, the thoracic flow signal will still show a small flow if the subject struggles to breath, even though no air is actually flowing. The flow is created because the subject exerts a pressure on the air which is always inside the lung. The pressure causes this air inside the lung to expand or contract. So, any change in internal lung pressure can be detected on the thoracic flow signal.

As airway resistance increases, the nasal flow is no longer in phase with the thoracic movement, with the thoracic flow waveform leading nasal airflow waveform by more and more because the thoracic flow waveform responds to the lung pressure which must develop before air starts to flow to the mouth. Thus, the magnitude of the time delay or phase shift is indicative of the extent of airway resistance.

The plethysmograph may be a double chamber plethysmograph in which the thoracic and nasal flows are recorded as separate signals. Other techniques, described in detail in the above Hoffman patents may also be used to separately acquire thoracic and nasal signals.

It is also known to collect respiratory data by placing the test subject in the test chamber of a whole body plethysmograph, such as the plethysmographs described in U.S. Pat. No. 5,379,777 to Lomask, issued Jan. 10, 1995, and U.S. Pat. No. 6,902,532 to Lomask, issued Jun. 7, 2005. As changes to the air volume within the test chamber occur, pressure variations are recorded by the transducer, which normally displays the recorded data in numerical form or as a graph.

Air volume within the test chamber can expand for only three reasons: 1) the air temperature increases, 2) a quantity of the enclosed air expands by reducing the pressure acting on it, or 3) matter within the chamber changes state to gas, i.e., water vapor is added to the gas by evaporation. There is essentially no volume change due to subject movement, because movement does not warm the air significantly nor does it cause a change of state. However, when the subject breathes, all of these events occur.

During inspiration, the air is warmed and humidified from the chamber temperature and humidity (e.g. 23 C at 30% humidity) to body temperature (e.g. 37 C) and saturated (i.e. 100% humidity) relative humidity at body temperature. In order to move the air into the lungs, a lower pressure within the lungs is created, causing the volume of air in the lung to expand. Also, state change occurs when the lungs provide water to humidify the air.

During expiration, much of the reverse is true, except the temperature change of the air is less. That is, the air is cooled by the nares only partially before it comes in contact with the air in the chamber. The temperature change of the expired air is from body temperature (about 37° C.) to some mid-temperature between body temperature and chamber temperature (˜30° C.). The humidity change is from saturation at body temperature to saturation at the exit mid-temperature. The temperature/humidity changes are significantly less than in inspiration. There is essentially no volume change due to temperature when warmer expired air leaves the subject and comes into contact with cooler chamber air because the cooler air is warmed as much as the warmer air is cooled. That is, the net heat change is zero.

The chamber air is continuously warmed by body heat, and with every expired breath. If the chamber were a perfect insulator, the subject would continually warm the air in the chamber until it reached body temperature. However, as the chamber air increases in temperature it begins to cool against the chamber walls. In addition, a bias flow through the chamber, which is necessary to remove CO₂ produced by the subject, removes some of the heat. These cooling effects balance the heat source within the chamber, and the chamber achieves an equilibrium temperature. The heat generated by respiration is balanced by a cooling over the respiratory cycle.

### SUMMARY OF THE INVENTION

One aspect of the present invention relates to the measurement of airway resistance using a single waveform that includes both thoracic and nasal data, such as is acquired from a whole body plethysmograph, instead of from separate thoracic and nasal waveforms. Since the entire subject in within a single chamber, the single waveform is determined by the combination of the thoracic and nasal flow of the test subject. It has been observed that the waveform changes in a characteristic fashion with changes in airway resistance: a peak appears to dominate a certain region of the waveform. Comparing that peak area to other regions of the waveform can be used to characterize airway resistance without the need to separately measure the thoracic and nasal flows.

Specifically, the Box Flow signal acquired from a Whole Body Plethysmograph is the unscaled difference between the nasal and thoracic flows. That difference will respond to the amplitude difference between the nasal and thoracic flows, and also to the phase shift between them. The effect of phase shift on the difference is most pronounced at the transitions from inspiration to expiration and from expiration to inspiration. There is a peak in the Box flow waveform at the transition from inspiration to expiration of the component waveforms, and an opposite-going peak at the transition from expiration to inspiration. These peaks can be related to the phase shift between the components of the composite waveform, and the phase shift has been shown to be related to specific airway resistance.

One index of specific airway resistance (Iₚᵣ) can be computed from three measured areas: an area during inspiration (A₂), an area during expiration (A₃), and the area of the negative peak that occurs between inspiration and expiration (A₁). The time duration of each of the three areas is identical. Time duration is calculated by measuring the time (Tₚ) from Box Flow zero at the beginning of A₁ to the Box Flow minimum, i.e., the negative apex, of A₁. The time duration is twice Tₚ.

The area during inspiration (A₂) is measured immediately before the zero crossing at the beginning of A₁. The area during expiration A₃ is measured after the Box Flow negative peak A₁. Specifically, A₃ begins at a time Tₚ past A₁. Areas A₂ and A₃ measure Box Flow during intervals when the Box Flow signal is dominated by temperature and humidity conditioning, not during intervals when the Box Flow signal may be impacted significantly by lung pressure changes.

The index of airway resistance (Iₚᵣ) may be expressed by the equation:

$${I}_{pr} = \frac{\vert {A}_{1} \vert \times 2 \times {T}_{p}}{\vert {A}_{2} \vert + \vert {A}_{3} \vert}$$

Functional Residual Capacity, FRC, which is defined as the volume of air that remains within the lung at the end of normal expiration, can also be estimated from the peaks at the transition from inspiration-to-expiration and expiration-to-inspiration. Specifically, FRC can be estimated from the ratio of the area of the peak from expiration-to-inspiration divided by the area of the peak from inspiration-to-expiration.

Another aspect of the present invention relates to the measurement of airway resistance from separate thoracic and nasal waveforms. In the procedure of the present invention, thoracic and nasal waveforms are acquired by known methods. The phase shift between the waveforms is then determined at two separate locations. The net inspired volume, i.e., the inspired volume minus the expired volume, between the two locations is determined, and the airway resistance is calculated from the phase shift and volume data.

Preferably, the phase shifts are measured at the transitions from expiration to inspiration and from inspiration to expiration. The dry gas pressure (atmospheric pressure minus vapor pressure) and respiratory rate are also measured.

Airway resistance can be calculated from the above data by dividing the difference in the tangents of the phase shifts times the dry gas pressure by 2Π×the respiratory rate×the net inspired volume. Thoracic gas volume can be calculated by dividing the inspired volume by the difference between the tangents of the phase shifts.

### BRIEF DESCRIPTION OF THE DRAWINGS

FIG. 1 is a graph of a single breath showing the nasal and thoracic flow.

FIG. 2 is graph of a single breath showing nasal and thoracic flow with increased airway resistance.

FIG. 3 is a graph of the difference in nasal and thoracic flows of the graphs of FIGS. 1 and 2.

FIG. 4 is a graph of a Box Flow signal illustrating calculation of the index of airway resistance.

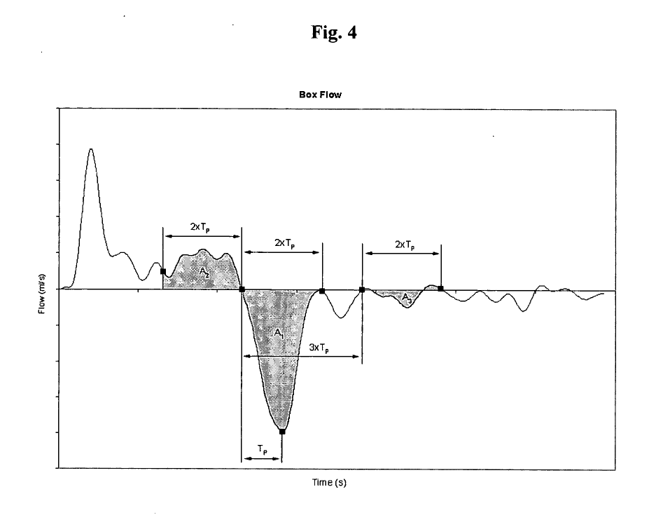

### DETAILED DESCRIPTION OF THE INVENTION

### Calculation of Index of Airway Resistance from a Single Waveform

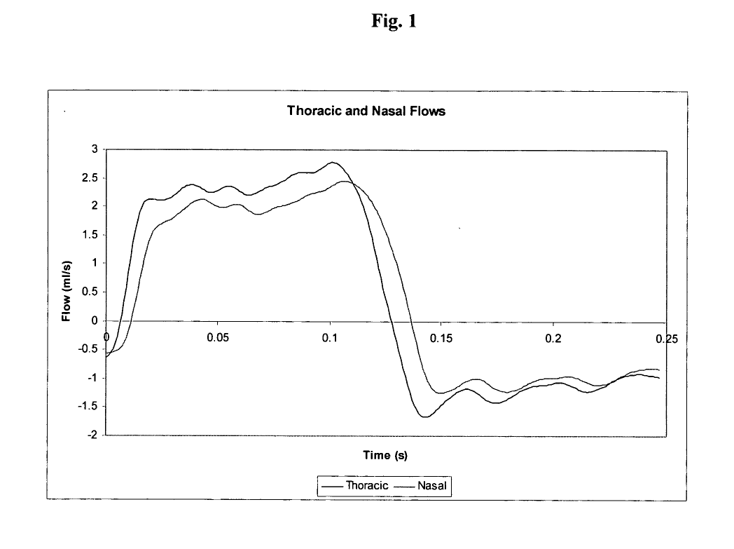

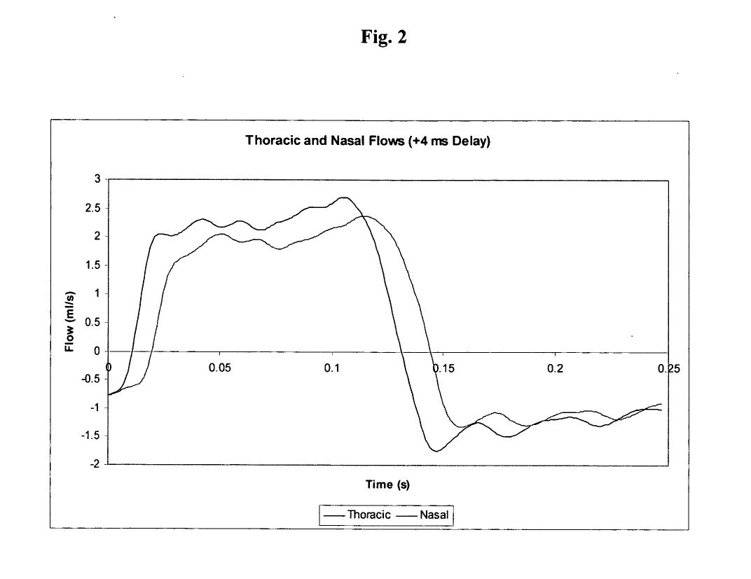

FIGS. 1 and 2 graphically illustrate the phase shift between separately acquired signals for nasal and thoracic flow resulting from airway resistance. Inspiration is positive and expiration is negative. The nasal flow signal has about the same shape as the thoracic flow signal, but is smaller and slightly delayed. FIG. 2 shows a longer delay between the nasal and thoracic flow signals, indicating increased airway resistance.

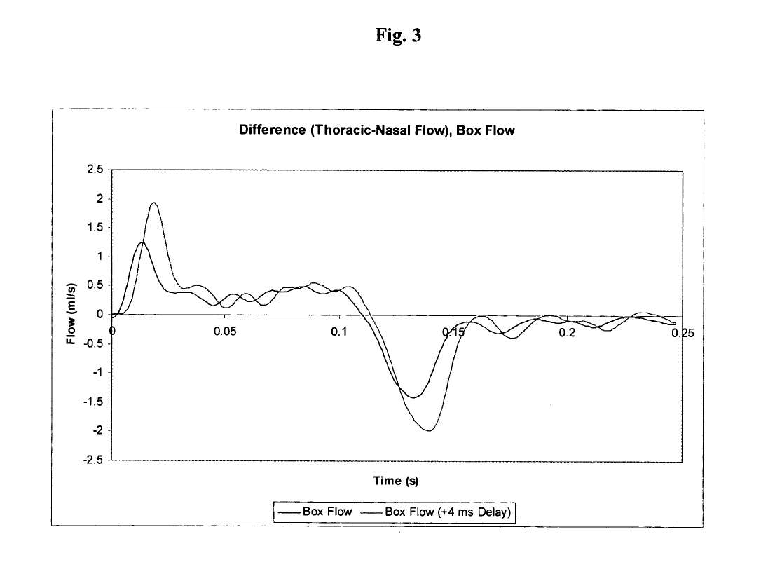

FIG. 3 shows the resulting Box Flow signals produced from the graphs in FIGS. 1 and 2. That is, the Box Flow signal (A) is produced from the flows shown in FIG. 1, and Box Flow signal (B) is produced from the flows shown on the graph in FIG. 2. Notice that Box Flow signal (B) has higher peaks and valleys which correlate with a longer delay.

One way to compute the phase shift between the nasal and thoracic flows is to first scale the nasal flow waveform so that its peak-to-peak magnitude is equal to the peak-to-peak magnitude of the thoracic flow. Then calculate a new waveform (scaled difference) by subtracting the scaled nasal flow from the thoracic flow. The time delay between the two flow signals can be measured by integrating a small region (small compared to a respiratory cycle, say 10% or less) on the scaled difference waveform, and dividing that result by the differences in thoracic flow from the start to the end of that integrated region. From the time delay, it is a simple matter to compute the phase shift. In summary, the scaled difference can be used to measure phase shift.

A phase shift can be calculated within almost any region of a single breath as long as the starting and ending flows are not equal. (If the starting and ending flows are the same, then the quotient will have a zero in the denominator.) However, some regions are better than others for practical computational reasons. For example, because a computer can represent a flow value along the signal with a specific finite number resolution, it is desirable that the starting and ending flow are as far apart as possible. This reason can also be applied to computing the difference between the nasal and thoracic flows. Assuming the phase shift is uniform, the difference between the nasal and thoracic flow is greatest where the slope of the flow signals is steepest.

As a result, the two best regions to measure the phase shift are regions surrounding the transition from inspiration-to-expiration and from expiration-to-inspiration. And since the subject may hesitate at the end of expiration, the transition from inspiration-to-expiration is best.

Since the Box Flow signal is the difference between the nasal and thoracic flows, it responds to changes in phase shift. And since it is the unscaled difference between the nasal and thoracic flows, it responds to amplitude difference between nasal and thoracic flows. A peak may be expected at the transition from inspiration-to-expiration due to the phase shift between the nasal and thoracic flows. We can also see a similar, but opposite-going peak at the transition from expiration-to-inspiration.

As described above, resistance information is readily available on the Box Flow signal at the transition from inspiration-to-expiration and from expiration-to-inspiration. This information is manifested by a peak surrounding that transition region. The area under this peak can be shown to be related to the developed pressure within the lung required to move the air either in or out. Also, the area under this peak is similar to the area computed between the nasal and thoracic waveforms in the double chamber application, which is an element in the computation of specific airway resistance. While not being purely related to resistance, or lung pressure, this peak is at least sensitively responsive to airway resistance.

### Example Index of Airway Obstruction

In order to calculate the index of airway resistance (Iₚᵣ) as a measurement of airway resistance from the peaks in graph (B) of FIG. 3, three areas are measured: an area during inspiration (A₂), and an area during expiration (A₃), and the area of the Box Flow negative peak which occurs between inspiration and expiration (A₁). The duration of each area is identical as determined by measuring the time (Tₚ) from the Box Flow zero to the Box Flow minimum within the negative peak. The duration is twice this measured time.

The area during inspiration (A₂) is measured immediately before the zero crossing. The area during expiration (A₃) is measured after the Box Flow negative peak (A₁). Specifically, A₃ begins Tₚ past A₁. The index of airway resistance is then measured in accordance with the following equation:

$${I}_{pr} = \frac{\vert {A}_{1} \vert \times 2 \times {T}_{p}}{\vert {A}_{2} \vert + \vert {A}_{3} \vert}$$

### Calculation of Functional Residual Capacity from a Single Waveform

Peak information can also be used to estimate functional resistance capacity (FRC). To estimate the subject's FRC, we start with the following equation, and simplify it:

$${\dot{V}}_{b} ( t ) \approx {\dot{V}}_{a} ( t ) ( 1 - \frac{{T}_{c , n} {P}_{a} ( t )}{{T}_{a} {P}_{c}} ) - {V}_{a} ( t ) {\dot{P}}_{a} ( t ) \frac{{T}_{c , n}}{{T}_{a} {P}_{c}}$$

### Where:

\dot{V}_{b}(t) is the flow of air out of the chamber (named the Box Flow),
\dot{V}ₐ(t) is the flow of air into the animal,
Vₐ(t) is the volume of air in the lungs,
T_{c} is the chamber temperature during inspiration,
Tₙ is the nasal temperature during expiration,
Tₐ is the subject's body temperature,
P_{c} is the dry air pressure within the chamber,
Pₐ(t) is the dry air pressure within the lungs, and

Making all these assumptions, if we integrate the peaks that occur, then we can estimate FRC as follows:

$$\begin{matrix}\frac{{Peak}_{Exp - to - Insp}}{{Peak}_{Insp - to - Exp}} \approx \frac{{K}_{2} ( FRC ) \int {\dot{P}}_{l} ( t ) d {t}_{Inspiration}}{- {K}_{2} ( FRC + {V}_{T} ) \int {\dot{P}}_{l} ( t ) d {t}_{Expiration}} \\ \approx \frac{FRC}{FRC + {V}_{T}} \\ = W\end{matrix}$$

W is the ratio of the area peak under each peak. The value can be easily derived from the box flow signal. And as shown above, this ratio is related to the ratio of the subject's pulmonary volume at the start of inspiration to the pulmonary volume at the end of inspiration.

With an estimation of V_{T} (tidal volume), FRC can be estimated by the following:

$$FRC = \frac{W • {V}_{T}}{W - 1}$$

### Estimating FRC and Airway Resistance Using Separate Nasal and Thoracic Flows

It is known from A Noninvasive Technique For Measurement Of Changes In Specific Airway Resistance, Pennock et al., J. Appl. Physiol.: Respirat. Environ. Exercise Physiol. 46(2): 399-406, (1979), that the following relationship is true:

tan θ=2πfR_{aw}C

where:
θ is the phase shift between the nasal and thoracic flows
C is the compressibility of the lung
R_{aw} is the airway resistance
f is the frequency of breathing

$$C = \frac{d V}{d P}$$

If the expansion or contraction is isothermal (and it is because it takes place at subject's body temperature), then the following relationship is true:

$$C = \frac{{V}_{tgv}}{{P}_{a}}$$

where:
V_{tgv} is the thoracic gas volume
Pₐ is the dry gas pressure in the lung
θ is the phase shift between the nasal and thoracic flows. This phase shift can be measured by determining the time (in seconds) that the nasal flow lags behind the thoracic flow and the frequency of breathing in Hertz.

θ=2πfd

where:
d is the time in seconds that the nasal flow lags behind the thoracic flow
f is the frequency of breathing
   Applying these other equations, we can rewrite the original equation:

$$tan θ = \frac{2 π {fR}_{aw} {V}_{tgv}}{{P}_{a}}$$

The thoracic gas volume (V_{tgv}) is different at the start of inspiration than it is at the end of inspiration. And this difference can easily be measured by integrating the thoracic flow signal. This value is routinely reported as the tidal volume (V_{T}).

$$tan {θ}_{start} = \frac{2 π {fR}_{aw} ( FRC )}{{P}_{a}}$$

$$tan {θ}_{end} = \frac{2 π {fR}_{aw} ( FRC + {V}_{T} )}{{P}_{a}}$$

Knowing these two equations, an equation can be derived both for FRC and R_{aw}.

To simplify the following derivations, substitute the tangent terms as follows:

$${D}_{start} = tan {θ}_{start}$$

$${D}_{end} = tan {θ}_{end}$$

$$FRC = \frac{{D}_{start} {P}_{a}}{2 π {fR}_{aw}}$$

$$\begin{matrix}{D}_{end} = \frac{2 π {fR}_{aw} [ \frac{{D}_{start} {P}_{a}}{2 π {fR}_{aw}} + {V}_{T} ]}{{P}_{a}} \\ = {D}_{start} + \frac{2 π {fR}_{aw} {V}_{T}}{{P}_{a}}\end{matrix}$$

$${R}_{aw} = \frac{[ {D}_{end} - {D}_{start} ] {P}_{a}}{2 π {fV}_{T}}$$

$$FRC = \frac{{D}_{start} {V}_{T}}{[ {D}_{end} - {D}_{start} ]}$$

Certain modifications and improvements will occur to those skilled in the art upon a reading of the foregoing description. It should be understood that all such modifications and improvements have been deleted herein for the sake of conciseness and readability but are properly within the scope of the following claims.

What is claimed is:

1. A method of characterizing the respiratory properties of a conscious living organism from a single respiratory waveform that includes thoracic and nasal flow signal components comprising:
a) acquiring a single box flow waveform that includes thoracic and nasal flow signal components;
b) measuring area of peaks of said waveform; and
c) characterizing respiratory properties from said peak areas.

2. The method of claim 1, wherein said waveform is acquired using a whole body plethysmograph.

3. The method of claim 1, wherein said area peaks include the area of the peak between inspiration and expiration.

4. The method of claim 1, including measuring an area of the signal during inspiration, measuring an area of the signal during expiration, and measuring an area of the signal between inspiration and expiration, said areas having the same time duration.

5. The method of claim 1, wherein said respiratory property is calculated as the index of airway resistance using the equation:
$${I}_{pr} = \frac{\vert {A}_{1} \vert \times 2 \times {T}_{p}}{\vert {A}_{2} \vert + \vert {A}_{3} \vert}$$
wherein A₁ is the area of the box flow negative peak between inspiration and expiration, A₂ is the area during inspiration is measured immediately before the zero crossing, A₃ is the area during expiration is measured at Tₚ after A₁.

6. A method of characterizing the respiratory properties of a conscious living organism as the index of airway resistance Iₚᵣ from a single respiratory waveform that includes thoracic and nasal flow signal components comprising:
a) acquiring a single box flow waveform that includes thoracic and nasal flow signal components;
b) measuring an area of the waveform during inspiration, measuring an area of the signal during expiration, and measuring an area of the signal between inspiration and expiration, said areas having the same time duration; and
c) calculating Iₚᵣ from the relationship and volumes of said areas.

7. The method of claim 6, wherein said waveform is acquired using a whole body plethysmograph.

8. The method of claim 6, wherein Iₚᵣ is calculated as the index of airway resistance using the equation:
$${I}_{pr} = \frac{\vert {A}_{1} \vert \times 2 \times {T}_{p}}{\vert {A}_{2} \vert + \vert {A}_{3} \vert}$$
wherein A₁ is the area of the box flow negative peak between inspiration and expiration, A₂ is the area during inspiration is measured immediately before the zero crossing, A₃ is the area during expiration is measured at Tₚ after A₁.

9. The method of claim 1, wherein said respiratory property is functional residual capacity, and said property is calculated as the ratio of the area of the peak from expiration to inspiration divided by the area of the peak from inspiration to expiration.

10. A method of characterizing the respiratory function of a conscious living subject comprising:
a) acquiring separate thoracic and nasal respiratory waveforms;
b) determining the phase shifts between said waveforms at first and second time spaced points;
c) determining the net inspired volume between said points; and
d) characterizing respiratory function using the phase shifts and net inspired volume.

11. The method of claim 10, wherein said first point is at the transition from expiration to inspiration.

12. The method of claim 10, wherein said second point is at the transition from inspiration to expiration.

13. The method of claim 10, wherein said respiratory function is characterized by dividing the difference in the phase shifts by the net inspired volume.

14. The method of claim 10, wherein said net inspired volume is equal to the total inspired volume between said points minus the total expired volume between said points.

15. The method of claim 10, further including the step of measuring the subject's respiratory rate and using said respiratory rate with said phase shifts and net inspired volume to characterize said respiratory function.

16. The method of claim 10, further including the step of measuring the subject's lung pressure and using said pressure measurement with said phase shifts and net inspired volume to characterize said respiratory function.

17. The method of claim 10, wherein the respiratory function is airway resistance (R_{aw}), characterized by the equation:
$${R}_{aw} = \frac{[ {D}_{end} - {D}_{start} ] {P}_{a}}{2 π {fV}_{T}}$$
wherein D_{end} is equal to tan θ_{end}, Dₛₜₐᵣₜ is equal to tan θₛₜₐᵣₜ, θ_{end} is the second phase shift, θₛₜₐᵣₜ is the first phase shift, Pₐ is the dry gas pressure in the subject's lung, f is the respiratory rate, and V_{tgv} is the thoracic gas volume.

18. The method of claim 10, wherein the respiratory function is functional residual capacity FRC characterized by the equation:
$$FRC = \frac{{D}_{start} {V}_{T}}{[ {D}_{end} - {D}_{start} ]}$$
wherein D_{end} is equal to tan θ_{end}, Dₛₜₐᵣₜ is equal to tan θₛₜₐᵣₜ, θ_{end} is the second phase shift, θₛₜₐᵣₜ is the first phase shift, and Vₜ is the tidal volume.

19. A method of characterizing the respiratory function of a conscious living subject comprising:
a) acquiring separate thoracic and nasal respiratory waveforms;
b) determining the a first phase shift between said waveforms at the transition from expiration to inspiration and a second phase shift between said waveforms at the transition from inspiration to expiration;
c) determining the net inspired volume between said first and second phase shifts; and
d) dividing the difference in the phase shifts by the net inspired volume.

20. The method of claim 19, further including the steps of measuring the subject's respiratory rate and the subject's lung pressure and using said respiratory rate and the pressure with said phase shifts and net inspired volume to characterize said respiratory function.
